(19) United States
(12) Patent Application Publication
(10) Pub. No.: US 2026/0016419 A1
(43) Pub. Date: Jan. 15, 2026

(54) Apparatus and Testing Methodology for Optically Determining Properties Specific to Drill Well Cuttings

(71) Applicant: Geolog S.r.l., San Giuliano Milanese (MI) (IT)

(72) Inventor: Antonio Calleri, San Giuliano Milanese (IT)

(21) Appl. No.: 19/262,586

(22) Filed: Jul. 8, 2025

(30) Foreign Application Priority Data
Jul. 9, 2024 (IT) 102024000015802

(51) Int. Cl. G01N 21/88; G01N 1/22; G01N 1/40; G01N 33/24

(52) U.S. Cl. CPC G01N 21/8806; G01N 1/2226; G01N 1/4044; G01N 33/241; G01N 2001/2241; G01N 2021/8835; G01N 2021/8845; G01N 2201/12761

(57) ABSTRACT

An apparatus includes an enclosure body providing an enclosed space eliminating external sources of electromagnetic radiation with a door and a sample plate; a camera device mounted to the enclosure body and including a lens, an image sensor, and a processing unit; and a lighting system mounted in the enclosure body and having at least a first set of illumination elements, the lighting system being configured to illuminate a sample arranged on the sample plate, wherein the camera device is configured to detect radiation resulting from an interaction between illumination provided by the lighting system and the sample.

### CROSS-REFERENCE TO RELATED APPLICATIONS

This application claims priority Italian Application No. 102024000015802, filed Jul. 9, 2024, which is incorporated herein by specific reference.

### DESCRIPTION

### Technical Field

The subject matter disclosed herein relates to analysis and testing devices applied to the Energy Industry. Particularly, the invention is related to an apparatus and testing methodology for optically and digitally determining properties specific to drill cuttings and rock samples extracted from boreholes and drilled wells.

### Background of the Disclosure

Petroleum geology relies on information gleaned through careful analysis of the geologic material excavated by a drilling operation. The cuttings contained in a drilling fluid provide detailed information about the geologic strata through which the drilling operation occurs.

As a part of routine analyses conducted on a drilling well, cutting samples are an available and informationally important resource for well-bore record creation. The information contained in the cutting sample(s) gives detailed information about the geology and potential fluid content of the borehole in which the analysis is performed. As an aspect of that analysis, specific parameters are of specific importance: color, particle size, particle shape, luminescence among others. The specific benefits and improvements obtained by the invention will be discussed and made clear in detail in the description of the invention.

Due to the complexities and subtle differences between geological formations within the sub-surface, non-biased or perfectly calibrated analysis of the rock cuttings can be difficult to perform by even specially trained scientists and geologists employed by companies in the sector. In different locations, or with different scientists, the analysis by a person may be biased or influenced in such a way as to obfuscate the small differences between lithologies in the drilling operation.

To remedy the non-repeatability of expert analysis, the invention seeks to automate and therefore overcome the issues of a qualitative manual assessment/analysis through consistency and repeatability. The robustness and repeatability of the invention disclosed permits analysis of rock cuttings by experts in location and operator-agnostic processes. This invention eliminates potential variations and creates consistent, robust and reliable analysis processes for further action.

### SUMMARY OF THE DISCLOSURE

As said, the disclosure refers to an apparatus and method for performing a standardized visual analysis on cuttings samples.

Preferably, the apparatus comprises an enclosure body providing an enclosed space.

Preferably, the enclosure body eliminates external sources of electromagnetic radiation.

Preferably, the enclosure body comprises an enclosure door.

Preferably, the apparatus comprises a sample plate.

Preferably, the apparatus comprises a camera device.

Preferably, the camera device is mounted to said enclosure body.

Preferably, the camera device comprises a lens.

Preferably, the camera device comprises an image sensor.

Preferably, the camera device comprises a processing unit.

Preferably, the camera device comprises an external connection means.

Preferably, the apparatus comprises a lighting system.

Preferably, the lighting system is configured to illuminate a sample arranged on said sample plate.

Preferably, said camera device is configured to detect radiation resulting from an interaction between illumination provided by said lighting system and said sample.

Preferably, the lighting system is mounted in said enclosure body.

Preferably, the lighting system comprises at least a first set of illumination elements.

Preferably, the first set of illumination elements produce white light.

Preferably, the lighting system comprises a second set of illumination elements, and wherein said second set of illumination elements produce ultraviolet light.

Preferably, the second set of illumination elements produce both UVA and UVC light.

Preferably, the apparatus comprises a safety switch attached to the enclosure body configured to sense the opening and closing of the enclosure door.

Preferably, the apparatus comprises an external device configured to control at least the camera device.

Preferably, the apparatus comprises an anti-vibration device.

Preferably, said anti-vibration device is coupled to the enclosure body.

Preferably, said anti-vibration device is configured to reduce or eliminate vibrations to which the enclosure body may be subjected.

Preferably, the apparatus comprises an access window.

Preferably, the access window is configured to allow for the introduction of a solvent to the sample.

Preferably, the solvent is selected to react with residual hydrocarbons contained in the sample.

Preferably, the method for utilizing the apparatus comprises a step of collecting and preparing a sample for arrangement inside the enclosure body.

Preferably, the method for utilizing the apparatus further comprises placing the prepared sample inside the enclosed space, on the sample plate.

Preferably, the method for utilizing the apparatus further comprises illuminating the sample using a lighting system.

Preferably, the method for utilizing the apparatus further comprises measuring the characteristics of the reflected electromagnetic radiation.

Preferably, the method for utilizing the apparatus comprises generating a report based on parameters computed from the measured characteristics of the reflected electromagnetic radiation.

Preferably, the method for utilizing the apparatus comprises performing a calibration before the collection and preparation of a sample.

Preferably, the calibration comprises placing inside the enclosure body a standard gray card.

Preferably, the calibration comprises illuminating and focusing the camera device.

Preferably, the calibration comprises capturing a plurality of test images at different parameter values.

Preferably, the calibration comprises processing the resulting test images to determine the optimal parameters for image collection in relation to said standard gray card.

Preferably, the calibration additionally includes the step of centering the Field of View.

Preferably, centering the Field of View comprises obtaining output from the camera device.

Preferably, centering the Field of View comprises comparing the center of a sample plate with the center of the Field of View for the camera device.

Preferably, centering the Field of View comprises adjusting a camera support to minimize the difference between the centers of the sample plate and the camera device field of view.

Preferably, the method for utilizing the apparatus further comprises generating a report based on parameters computed from the collected image or images.

Preferably, the apparatus can be set to record video footage.

Preferably, the apparatus can be set to switch automatically between lighting conditions.

Preferably, the method allows the apparatus to automatically switch between image capture and video recording modes.

Preferably, the apparatus further comprises an enclosure fan configured to extract solvent fumes from inside the enclosure.

### BRIEF DESCRIPTION OF THE DRAWINGS

The accompanying drawings serve as further illustration of the invention. The figures represent one or more embodiments which, together with the detailed description, serve to define embodiments of the invention.

FIG. 1 is a schematic showing a particular and exemplary structure of the apparatus as viewed from the inside.

FIG. 2 is a schematic showing the external aspects of the apparatus along with elements mounted on the same.

FIG. 3 is a block diagram showing an exemplary embodiment of the sensory capabilities of the apparatus.

FIG. 4 is a schematic front view of an embodiment of the present invention.

FIG. 5 is a schematic plant view of some components of the embodiment of FIG. 4.

FIG. 6 is a schematic side view of a component of FIG. 5.

FIG. 7a is a schematic side, exploded view of the component of FIG. 5.

FIG. 7b is a schematic plant view of the parts shown in FIG. 7a.

### DETAILED DESCRIPTION OF THE PREFERRED EMBODIMENTS

In the following description, usage of the word “light” is to be interpreted as electromagnetic radiation. Further clarification on the part of the spectrum of interest with visible light denoting the visible spectrum and ultraviolet (UV) or infrared (IR) as their similarly associated spectrum bands.

In the accompanying drawings, reference 1 generally designates an apparatus according to the present invention.

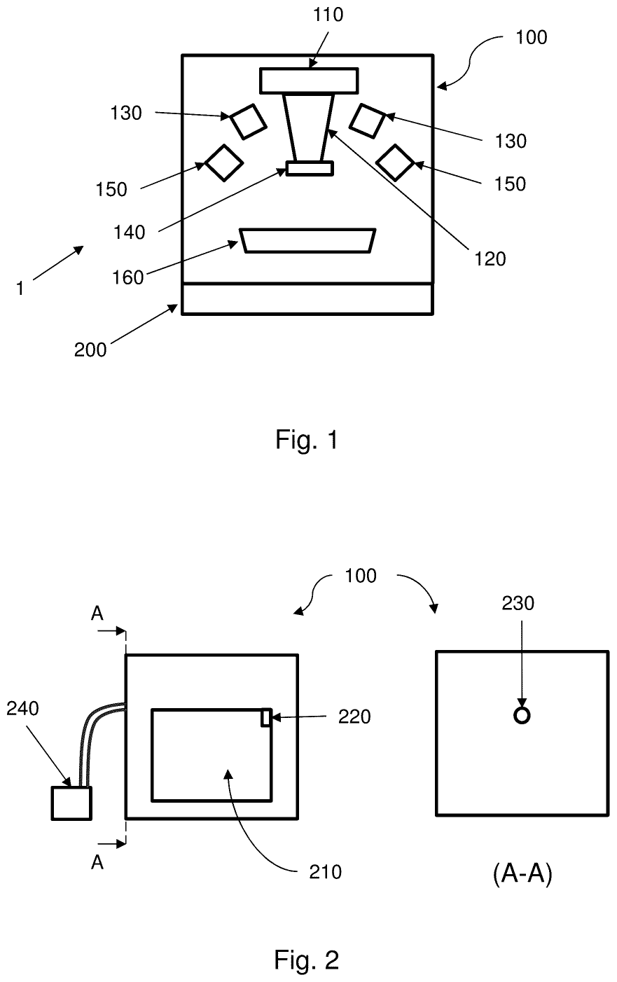

FIG. 1 shows an exemplary embodiment of the invention. Apparatus 1 shown in FIG. 1 comprises an enclosure body 100 which serves to encapsulate the internal elements and protect a sample of interest from external factors. The main external factor at issue is electromagnetic radiation. The enclosure body must be able to eliminate the incursion of incidental electromagnetic radiation to facilitate the measurements and subsequent analyses to be performed. The enclosure body 100 allows for the internal mounting of the additional elements of the invention. The enclosure body 100 may contain an internal shelf mounted in the upper section.

Located inside the enclosure body 100, a camera device 110 is mounted to the structure. The mounting may allow for variations in the centering of the camera device 110 in the enclosure body 100.

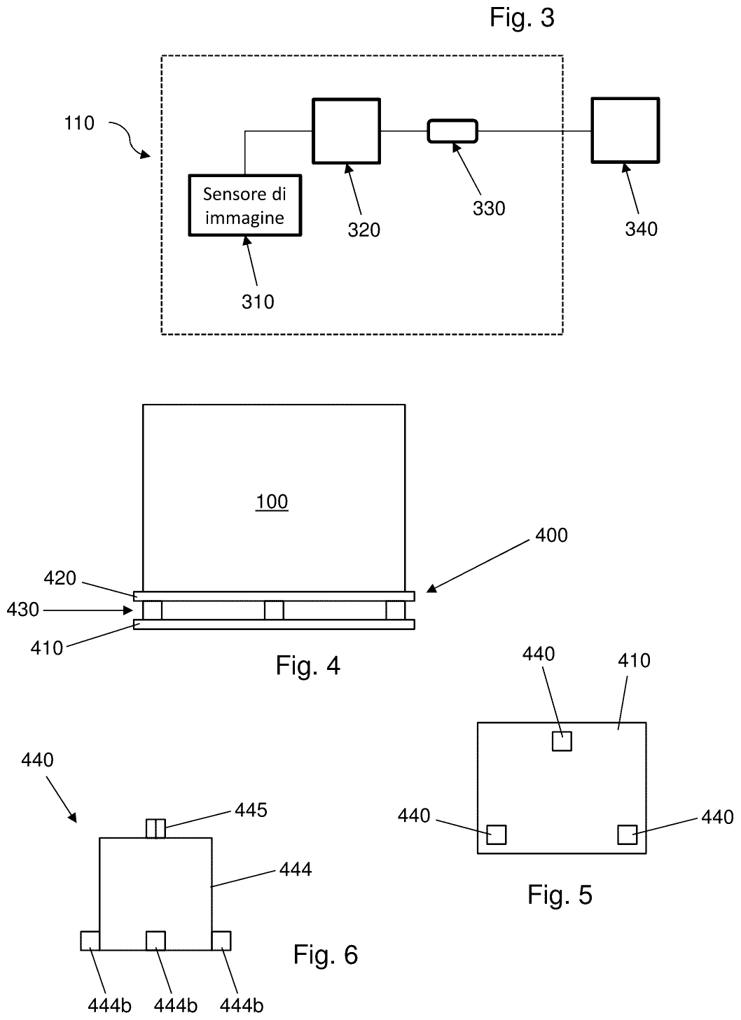

Lens 120 is an optical lens which enables a digital image sensor 310 (schematically shown in FIG. 3) to capture enough detail to perform further processing and analysis. Due to the general small size of the particles being photographed, the lens 120 plays a major role in the quality of the images gathered. The lens 120 is preferably a macro lens.

For example, the camera device 110 is a single-lens reflex (SLR).

For example, the camera device 110 is a hyper-spectral camera.

Due to the lack of external electromagnetic light sources, the invention provides controlled light sources for the accurate analysis of the samples. Apparatus 1 therefore comprises a lighting system 130, 140, 150 to provide the light for quality image collection. The lighting system comprises at least a first set of illumination elements 130 which provide visible light to illuminate the samples inside the enclosure body 100. Preferably, the first set of illumination elements 130 provide white light within a temperature range of 6000-6500 K. This range corresponds to limited impact of the provided light in biasing the calibration of the system. Preferably, the illumination elements 130 are located on either side of the camera device 110 to avoid shadows present in the sample.

The lighting system may also comprise a second set of illumination elements 140, 150. The second set of illumination elements 140, 150 serves to illuminate the sample with additional wavelength bands on the electromagnetic spectrum. Of particular interest in petroleum geology is ultraviolet fluorescence. The second set of illumination elements 140, 150 may optionally comprise ultraviolet elements. In a particular embodiment, the second set of illumination elements 140, 150 comprises two sets of ultraviolet elements, namely a first set of ultraviolet elements 140 and a second set of ultraviolet elements 150. The illumination of the sample with ultraviolet light can be then sensed by either an image sensor capable of sensing ultraviolet radiation or an image sensor configured to sense the fluorescence of specific elements in visible light under ultraviolet excitation.

The first set of ultraviolet elements 140 preferably outputs UVA light, preferably having a wavelength substantially equal to 365 nm. The first set of ultraviolet elements may take the form of a ring light as shown in FIG. 1, a pair of lamps flanking the camera device, or any other suitable configuration for illuminating the sample being imaged.

The second set of ultraviolet elements 150 preferably outputs UVC light, preferably having a wavelength substantially equal to 254 nm. The elements may optionally use a bandpass filter to produce light in the UVC band. The second set of ultraviolet elements 150 may be positioned in a similar manner to the first set of ultraviolet elements 140, preferably as a pair of lamps flanking the camera device 110.

In an embodiment, the lighting system 130, 140, 150 can be provided with a manual regulation module, through which an operator can activate/deactivate and regulate the lighting system and parts thereof, such as the first set of illumination elements 130 and the second set of illumination elements 140, 150.

In an embodiment, the lighting system 130, 140, 150 may be configured so as to switch automatically between lighting conditions. The switch may occur in conjunction with the imaging performed with the camera device 110.

Located inside the enclosure body 100 is a sample plate 160. The sample plate 160 is preferably made of stainless steel or porcelain and has squared and raised edges, creating a sample area in the center. The sample area is preferably square and has a size adapted to the sample size of cuttings coming from a well analysis. In a particular embodiment, the sample plate 160 also contains a center marking denoting the center of the plate. This central marking is provided to enable field-of-view (FOV) centering on each sample.

As schematically shown in FIG. 1, the camera device 110 is mounted to a top portion of the enclosure body 100. The lens 120 is interposed between the camera device 110 and the sample plate 160—the latter being arranged in a bottom portion of the enclosure body 100. The first set of ultraviolet elements 140, for example, can surround a lower portion of the lens 120, or be mounted close to the lower portion of the lens 120, so that the camera device 110 can detect the sample arranged on the sample plate 160, via the lens 120 and through the hollow space within the ring defined by the first set of ultraviolet elements 140.

Apparatus 1 is preferably a portable apparatus. The enclosure body 100 can be substantially box-shaped. For example, the enclosure body 100 can be cube-shaped or parallelepiped-shaped. Each side of the enclosure body, for example, can be 0.50 m to 0.80 m long.

FIG. 2 schematically shows a front and side view of apparatus 1. Enclosure door 210 blocks electromagnetic radiation while allowing a user to handle samples placed on the sample plate 160. Enclosure door 210 is also necessary to protect apparatus users from the UV elements 140, 150. In order to control the usage of second set of illumination elements, the enclosure body 100 also contains a safety switch 220 mounted and configured to sense the opening and closing of the enclosure door 210. The safety switch 220 provides a control for both a safety check as well as a parameter check for complete darkness created inside the enclosure after the placement of a sample on the sample plate 160. The safety switch 220 may comprise a typical mechanical or electrical switch designed to allow or prevent the activation of the various lighting systems or processing/analysis method.

Apparatus 1 may optionally comprise an access window 230, as shown in side view (A-A). Access window 230 may be a small opening placed in a side of the enclosure 100. The side of the enclosure 100 may be any of the available side walls so as to allow access to the sample plate 160 for a pipette, straw, or other similar apparatus. The pipette may preferably introduce a solvent to the sample. Access window 230 may take the shape of a circle, ellipsis, square, or similar and have an associated plug or cover which can, similarly to the enclosure door 210, block all extraneous light from entering enclosure 100 when closed. In a preferred embodiment of the invention, the associated plug is made of rubber to ensure a tight seal.

The pipette, straw, or similar apparatus may be used to introduce a solvent to the sample plate 160. The solvent may be selected from several solvents which react with residual hydrocarbons trapped or otherwise contained in the sample. In particular, Acetone may be used as the solvent. The solvent quantity is minimal, typically between 1 and 10 mL. In a particular embodiment, 5 mL of solvent is used.

In case of solvent addition, fumes may arise from interactions between the residual hydrocarbons and the solvent. To mitigate the fumes effects on the internal components, an enclosure fan 240 may be provided to extract solvent fumes. The removal of the solvent fumes may also prevent degradation of plastic or rubber components included with apparatus 1. The enclosure fan 240 can be connected to enclosure 100 in such a way as to mitigate the ingress of external light.

In a particular embodiment, the connection may be through a duct or curved connection to help mitigate light pollution. Enclosure fan 240 does not need to be particularly large and will only be utilized when solvent is introduced to the sample. In operation, due to the small amounts of solvent used, enclosure fan 240 may run between 1 to 3 minutes, a typical amount for the quantity of fumes produced.

Optionally, enclosure fan 240 and enclosure 100 may be connected via the access window 230. This connection may be a hinged door or similar contraption which allows the access window 230 to serve both purposes: open access to the sample plate 160 to deposit the solvent, and a connection to enclosure fan 240 for subsequent ventilation. In this case, a switch may be included to determine if the access window 230 closes, indicating the insertion of the solvent and programmed to send a signal to subsequently run the enclosure fan.

FIG. 3 shows a basic schematic diagram representing the components of the camera device 110. The camera device 110 comprises several different components, mainly: an image sensor 310, a processing unit 320, and an external connection means 330.

The image sensor 310 may be any form of digital image sensor capable of detecting incident electromagnetic radiation. The digital image sensor 310 may sense electromagnetic radiation over a large range of the electromagnetic spectrum not limited to visible light. Optionally, the image sensor 310 may sense ultraviolet light. In a particular embodiment, the image sensor 310 is a commercially available charge-coupled device (CCD) or CMOS sensor.

The processing unit 320 accepts and processes the raw image information from the image sensor 310 and creates a digital representation of the sample being analyzed. The data contained in the processed image created by the image sensor 310 comprises mainly color space information and light intensity for each of the pixels present on the image sensor 310. The processing unit 320 may perform conversions of the raw color data to a defined or standard color space like sRGB or similar. The processing unit 320 may be configured to allow for simultaneous transmission of images for analysis or calibration through the external connection means 330.

The external connection means 330 allow for the connection between the camera device 110 and an external device 340 for the transmission of image information to the external device 340 for analysis and/or calibration. The connection created between the camera device 110 and an external device 340 may be wired or wireless. The connection may be a USB connection or a network connection utilizing Wi-Fi, Bluetooth, or similar wireless connection with a radio frequency transceiver.

The external device 340 may be provided for enhanced control of apparatus 1 and/or further processing of the image information output by the camera device 110. The external device 340 may be a laptop computer, handheld computer device like a tablet, or the like. The external device 340 is configurable to connect to the camera device 110 through the external connection means 330 with appropriate ports or capabilities. As said, the connection can be through a physical USB connection or a wireless connection such as WiFi, Bluetooth, or other similar radio frequency transmission standards.

For example, the external device 340 can carry out analysis disclosed in co-pending U.S. patent application Ser. No. 18/358,816 in the name of the same Applicant (US Publication No. US 2024/0036023), herein incorporated by reference.

In an embodiment, the external device 340 may also provide control to the lighting system. The external device 340 may also provide processing logic to control the sequence and selection of the lighting system in tandem with the camera device.

Apparatus 1 is preferably properly calibrated before the complex analyses can be performed on the sample cuttings. Calibration allows for the repeatability, interchangeability, and interoperability of the resulting analysis across drilling operations around the world. After shipment of apparatus 1 to a testing site, several issues may present themselves. Mainly, the camera device 110 may become uncentered, and the lighting characteristics may vary.

The centering of the camera device 110 field-of-view (FOV) is preferably performed after every transportation of apparatus 1. Centering the FOV comprises obtaining an output from the camera device 110, comparing the center of a sample plate 160 with the center of the FOV, and adjusting the camera device 110 support to minimize the difference between the FOV and the center of the sample plate 160.

Obtaining output from the camera device 110 may occur by connecting the camera device 110 to the external device 340 by the external connection means 330, thereby transmitting a number of images from the camera device 110 to the external device 340. The output from the camera device 110 may preferably be a live video stream or a plurality of still images. Preferably, the camera device 110 can switch automatically or be controlled to switch automatically between a video stream and still images.

Comparing the center of the camera FOV and the center of the sample plate 160 occurs by comparing the center of the output from the camera device 110 and the center marking on the sample plate 160.

The camera device 110 mounting inside the enclosure body 100 may be a mounting bracket attached to the camera body itself through a standard socket as known in the art or attached by a mount connected directly to the lens 120. The mounting is then loosened and tightened respectively to make adjustments to center the center marking on the sample plate 160 within the output transmitted by the camera device 110.

After a centering of the FOV has been completed and at defined intervals of use, a calibration of the images is preferably performed to validate the quality of the analyses conducted. The calibration interval may vary according to specific needs. In a particular embodiment, the calibration is performed at the beginning of a new well or approximately once every 2 months of use, whichever is sooner. The calibration comprises the base steps of placing a standard card inside the enclosure, illuminating the enclosure and focusing the camera device, capturing a plurality of images with different parameter values, and processing the resulting images to determine setting values which match the standard image.

In a particular embodiment, the standard card is a standard 18% reflectance gray card. This card is provided as it is a standard value halfway between total black and total white, which, when capturing an 8-bit image, has ideal brightness and RGB values of 127.5. Additional cards may be provided depending upon the type and bit-size of the image.

Focusing the view of the camera device 110 may comprise a number of sub-steps. First, utilizing the image transmission the camera is focused onto the standard card. In a particular embodiment, the focusing step may be completed automatically or manually to focus the image. The focus may be improved by providing a sheet of scrap paper to allow the autofocus to work. Depending upon methods of autofocus, the scrap paper allows for image variations to serve as edges on which to focus the camera.

The parameter values of the image sensor 310 are then adjusted for a plurality of images taken. In a particular embodiment, the varied parameters comprise iso sensitivity and shutter speed. The parameters may also include white balance, focal length, or other camera parameters. The collection of the plurality of images occurs with multiple rounds of photos taken at each parameter setting. In a particular embodiment, the starting parameters comprise a shutter speed that varies between 1/15″ and 1/25″ seconds with an iso set at 400.

Upon collection of the plurality of images, the resulting images are processed to determine optimal parameters for image collection. The processing takes the form of an extraction of RGB and Brightness values (for example, as disclosed in the aforementioned U.S. patent application Ser. No. 18/358,816) which are averaged over all sets of images and a report of the values generated based on pixel values contained in the plurality of images. The closest match of the brightness value to the standard card value is selected as the optimal parameters for the apparatus.

Operation of the apparatus after calibration is comprised of the steps of collecting and preparing a sample for arrangement inside the enclosure body, placing the prepared sample inside the enclosed space, illuminating the sample using a lighting system, and measuring the characteristics of the reflected electromagnetic radiation.

The collecting and preparing step comprises placing onto the sample plate 160 a desired sample of cuttings. Cuttings may be obtained from a drilling operation or from a repository specifically designed to store such sample material. In a preferred embodiment, the cuttings may be in the range of 10-50 g. With a sample size generally less than 20 g, a centering tool may be utilized, as the sample may not fully cover the sample plate 160. The cuttings sample may be placed on the sample plate 160 in a way that the distribution of particle size is randomized. Excessive shaking may cause cuttings of smaller dimensions to self-separate, biasing a subsequent analysis. The cuttings are wetted to improve image acquisition. In a particular embodiment the wetting is performed with a set volume of deionized water. The wetting step is performed with a standardized spray bottle positioned at a distance above the cutting sample in the sample plate. The proper distance being preferably between 15-20 cm perpendicular to the sample plate. A minimum number of sprays should be used to avoid oversaturation of the cuttings which reflect unevenly the light coming from the lighting system.

The placement step comprises placing the wetted sample into the enclosure body 100. The placement can be controlled by any centering device 200 located at the bottom of the enclosure body. In a particular embodiment, the placement of the sample in the enclosure body 100 is centered using a set of rails adapted to the sample plate 160 dimensions, where the rails terminate at the optimal location for the sample plate 160. The sample is centered when the sample plate 160 is abutting the end of the set of rails. The enclosure door may then be closed to avoid external light sources.

The illuminating and measuring steps are linked together. The illumination occurs in at least a first stage. The first stage of illumination occurs utilizing the lighting system with the first set of illumination elements 130. In a particular embodiment, the illumination also comprises a second stage where the illumination occurs utilizing the lighting system with the second set of illumination elements 140, 150. When enclosed within the apparatus, images are obtained which measure the reflected or luminated light from the lighting system. The measurement is performed with the image sensor 310 provided by the camera device 110.

In an embodiment, a Focused Image stacking technique is applied to the images taken. Focused Image stacking combines a group of images with a similar frame of reference, but taken at different focal planes, into one single image. Such technique appears advantageous in the present context, as 3D objects of different sizes and shapes (cuttings particles) are involved and, for the final image, all objects need to be captured in focus with sharp edges and clear definition.

In a particular embodiment, the measurement provided by the image sensor 310 is used to generate a report describing the characteristics of the sample. The measurement may be a tensor of pixel data from the image sensor or a processed image file obtained from the processing unit 320. The processed image file may be of a known image file type, including but not limited to raw, jpg, tiff, or the like. The generated report may include details regarding the cuttings physical parameters, rock type, or other discernable information related to a visual analysis of the sample. Specifically, the analysis may include statistical parameters related to cutting size, shape, composition, color, or other related value, possibly combined with data obtained from X-ray fluorescence (XRF), X-ray diffraction (XRD), Laser Induced Breakdown Spectroscopy (LIBS) hyper-spectral analysis or analysis of extracted hydrocarbons contained within the sample.

In an embodiment, apparatus 1 is provided with an anti-vibration device 400 (FIGS. 4-6, 7a-7b).

The anti-vibration device 400 is arranged at the bottom of the enclosure body 100.

More in general, the anti-vibration device 400 is arranged between the enclosure body 100 and the wall/surface to which the enclosure body 100 is constrained.

For example, apparatus 1 can be arranged on a table or on the floor (or on the ground). Accordingly, the anti-vibration device 400 will be located between the enclosure body 100 and the table/floor/ground. The weight of apparatus 1 may create the constraint between the same apparatus 1 and the table/floor/ground.

It is worth emphasizing that apparatus 1 is conceived to be employed “in-field”, i.e., at the rig or in the vicinity thereto. Thus, the provision of the anti-vibration device 400 allows to achieve important advantages in terms of improvement of accuracy and precision of the analysis carried out based on the detections made inside the enclosure body 100.

Preferably, the anti-vibration device 400 comprises a first wall 410 and a second wall 420 (FIG. 4).

The first wall 410 is configured to be constrained to some wall/surface; for example the first wall 410 can be placed on a table or on the floor/ground.

The second wall 420 is attached to a wall (e.g., the bottom wall) of the enclosure body 100.

Preferably, the second wall 420 is substantially parallel to the first wall 410.

The anti-vibration device 400 comprises a cushioning structure 430, interposed between the first wall 410 and the second wall 420.

The cushioning structure 430 advantageously provides a mechanical decoupling between the first and second walls 410, 420, so that vibrations experienced by the first wall 410 and not transmitted to the second wall 420—or, at least, are significantly reduced.

In an embodiment, the second wall 420 coincides with the corresponding wall (e.g., the bottom wall) of the enclosure body 100. In other words, in this embodiment the second wall 420 is comprised in the enclosure body 100.

The cushioning structure 430 can comprise one or more cushioning elements 440.

In the example shown in FIG. 5, the cushioning structure 430 comprises three cushioning elements 440.

Each cushioning element 440 performs an anti-vibration function along a first direction (i.e., a direction substantially perpendicular to the second wall 420) and second directions (i.e., directions parallel to the second wall 420). Imagining that apparatus 1 rests on a substantially horizontal table, the first direction is a vertical direction, whereas the second directions are horizontal directions.

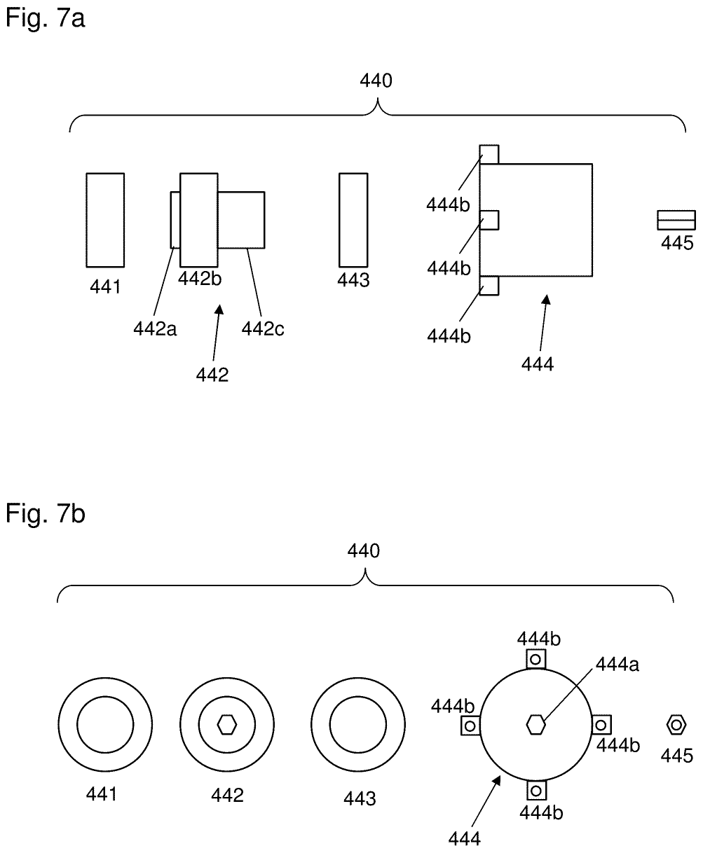

In an embodiment, each cushioning element 440 comprises (FIGS. 6, 7a-7b):
a first deformable ring 441, preferably made of elastomeric material (e.g., polyurethan foam);
a structural element 442, preferably made of a rigid material (e.g., steel), the structural element 442 having a substantially cylindrical shape, with a radially enlarged portion 442b located between a first axial end portion 442a and a second axial end portion 442c; the first deformable ring 441 is fitted on the first axial end portion 442a of the structural element 442. The structural element 442 is provided with a central, axial cavity 442d, which has a non-circular profile; FIG. 7b shows an exemplary hexagonal profile, however a square, rectangular, elliptical, etc. profile might be used;
a second deformable ring 443, preferably made of elastomeric material (e.g., polyurethan foam); the second deformable ring 443 is fitted on the second axial end portion 442b of the structural element 442;
a cover member 444, preferably made of rigid material (e.g., steel), adapted to enclose the first deformable ring 441, the structural element 442, and the second deformable ring 443. The cover member 444 can generally have a cylindrical shape, open at one end (so as to allow insertion of the other elements comprised in the cushioning element 440), and close at the opposite end. At such opposite end, a through opening 444a is provided, having the same shape as the cavity 442d of the structural element 442. The diameter and height of the cover member 444 are approximately equal to the diameter and the height, respectively, of the assembly formed by the first deformable ring 441, the structural element 442 and the second deformable ring 443. The cover member 444 is preferably provided with one or more engagement protrusions 444b (for example, four engagement protrusions 444b, each spaced by 90° from the adjacent ones), for mounting on the first wall 410;
a connection rod 445, having a cross-section corresponding to the profile of the cavity 442d; the connection rod 445 allows for connection with the second wall 420 and, more generally, with the structure with respect to which an anti-vibration function is to be provided—in the case of the present invention, such structure is the enclosure body 100.

In an embodiment, the anti-vibration device 400 can include side walls (not shown), connecting the first wall 410 with the second wall 420 so as to form a box structure in which the cushioning structure 430 (i.e., the cushioning elements 440) is housed.

1. An apparatus comprising:
an enclosure body providing an enclosed space eliminating external sources of electromagnetic radiation with a door and a sample plate;
a camera device mounted to said enclosure body and comprising a lens, an image sensor, and a processing unit; and
a lighting system mounted in said enclosure body and comprising at least a first set of illumination elements, the lighting system being configured to illuminate a sample arranged on said sample plate,
wherein said camera device is configured to detect radiation resulting from an interaction between illumination provided by said lighting system and said sample.

2. The apparatus according to claim 1, wherein said first set of illumination elements produce white light.

3. The apparatus according to claim 2, wherein said lighting system further comprises a second set of illumination elements, and wherein said second set of illumination elements produce ultraviolet light.

4. The apparatus according to claim 3, wherein the second set of illumination elements has both UVA and UVC wavelengths.

5. The apparatus according to claim 4, wherein the apparatus further comprises a safety switch attached to the enclosure body and configured to sense the opening and closure of the enclosure door.

6. The apparatus according to claim 1, further comprising a processing device configured to control at least said camera device and automation of the lighting settings.

7. The apparatus according to claim 1, further comprising an anti-vibration device, coupled to the enclosure body and configured to reduce or eliminate vibrations to which the enclosure body may be subjected.

8. The apparatus according to claim 7, further comprising an access window configured to allow for the introduction of a solvent to said sample, said solvent selected to react with residual hydrocarbons therein contained.

9. The apparatus according to claim 8, further comprising an enclosure fan configured to extract solvent fumes from inside the enclosure.

10. A method for utilizing an apparatus according to claim 1, comprising:
collecting and preparing a sample for arrangement inside the enclosure body;
placing the prepared sample inside the enclosed space on the sample plate;
illuminating the sample using said lighting system; and
measuring characteristics of the reflected electromagnetic radiation.

11. The method according to claim 10, wherein the step of collecting and preparing a sample comprises spraying a collected sample with water.

12. The method according to claim 11, wherein a calibration is performed before said collection and preparation of a sample, said calibration comprising:
placing inside the enclosure body a standard gray card;
illuminating and focusing the camera device;
capturing a plurality of test images at different parameter values; and
processing the resulting test images to determine the optimal parameters for image collection in relation to said standard gray card.

13. The method according to claim 12, wherein the calibration, when performed after transportation, additionally includes the step of centering the Field of View, centering the Field of View comprising:
obtaining output from the camera device;
comparing the center of a sample plate with the center of the Field of View for the camera device; and
adjusting a camera support to minimize the difference between the centers of the sample plate and the camera device field of view.

14. The method of claim 13, further comprising generating a report based on parameters computed from the measured characteristics of the reflected electromagnetic radiation.

15. The method of claim 14, further comprising generating a report based on parameters computed from the collected image or images.
